(19) United States
(12) Patent Application Publication
(10) Pub. No.: US 2008/0226019 A1
(43) Pub. Date: Sep. 18, 2008

(54) Multiple Scatter Correction

(75) Inventors: Axel Thran, Hamburg (DE); Jens-Peter Schlomka, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/994,460

(22) PCT Filed: Jul. 6, 2006
(86) PCT No.: PCT/IB06/52281

§ 371 Date: Jan. 2, 2008

(30) Foreign Application Priority Data
Jul. 7, 2005 (EP) 05106197.6

(51) Int. Cl. A61B 6/00; G01N 23/201

(52) U.S. Cl. 378/7; 378/86

(57) ABSTRACT

According to an aspect of the present invention, a correction of X-ray intensities measured in an energy-resolved diffraction method may be provided for multiple scattered radiation without any assumptions on the geometry of the object examined. According to an exemplary embodiment of the present invention, the characteristic lines of the anode material in the primary spectrum are evaluated, resulting in a component analysis of the detected spectrum which may allow for a correction for its multiple scatter part.

The present invention relates to the field of X-ray imaging. In particular, the present invention relates to an examination apparatus for examination of an object of interest, to a method of examining an object of interest with an examination apparatus, an image processing device, a computer-readable medium and a program element.

X-ray scatter techniques generally aim to detect the scatter function of a material or of an object part. Only single-scattered photons contribute to an evaluable signal. Multiple scattered photons do not contain valuable information and generally form background signal impairing the measurement. The larger the object the higher is the probability for a photon to scatter several times in the object. Therefore, a correction of the multiple scatter intensity is necessary in particular for objects much larger than the scattering mean free path of the X-ray photons.

Coherent scatter computed tomography (CSCT) is a new imaging technique based on coherently scattered X-ray photons. A collimated fan-beam of small divergence out of the fan plane exposes an object. Both signals, the intensity of the transmitted radiation and the intensity of the scattered radiation caused by scatter processes within the object, are measured. As in a CT scanner a multitude of projections with different rotational positions are measured. Similar to reconstruction of images in CT the scatter function of each point in the illuminated object area can be reconstructed from measured scatter projections.

According to Monte-Carlo simulations, typically about half of the scatter intensity measured in the CSCT projection of a 20 cm thick water phantom is multiply scattered. The amount of multiple scattered radiation depends not only on the penetrated object thickness and the object material but also on the extension of the object perpendicular to the fan plane. Thus, a correction without any additional knowledge of the object is difficult.

It may be desirable to have an improved correction for multiple scatter intensities.

According to an exemplary embodiment of the present invention, an examination apparatus for examination of an object of interest may be provided, the examination apparatus comprising a radiation source adapted for emitting electromagnetic radiation, an energy resolving detector unit having at least one detecting element for acquisition of radiation intensity data, and a pre-processing unit, the pre-processing unit being adapted for determining a first multiple scatter intensity in a vicinity of a characteristic peak of the radiation intensity data, and correcting the radiation intensity data on the basis of the first multiple scatter intensity.

However, it should be noted that the pre-processing unit may be separated from a reconstruction unit or integrated in a reconstruction unit.

Therefore, according to this exemplary embodiment of the present invention, a correction of X-ray intensities measured with an energy-resolving detector for multiple scattered radiation may be performed without any assumptions on the geometry of the object of interest.

Thus, a multiple scatter correction may be provided without any additional knowledge of the object of interest. This correction may provide for an improved image quality.

According to another exemplary embodiment of the present invention, the detector unit comprises a first detecting element and a second detecting element, wherein the determination is performed for the first detecting element and the second detecting element.

Therefore, an individual first determination of the first multiple scatter intensity at a certain energy only may be performed for a plurality of single, energy resolving detecting elements of the detector unit. This may improve the quality of the resulting image.

According to another exemplary embodiment of the present invention, the correction is performed on the basis of a second estimation of a second multiple scatter intensity over the full measured energy range for each first and second detecting element of the detector unit. Furthermore, the radiation intensity data acquired by the detector unit comprises transmitted intensity data, wherein the estimation is performed on the basis of the first multiple scatter intensity and the transmitted intensity data.

As well in transmission imaging as in X-ray scattering multiple scattered photons lead to a contribution in the detector intensity which is of no value for the measurement but leads to artifacts. Therefore a correction of the intensity originating from multiply scattered photons eliminates the corresponding artifacts and improves the image or data quality.

According to another exemplary embodiment of the present invention, the characteristic peak is a characteristic line of an anode material of the radiation source.

Therefore, the amount of multiple scattered radiation may be determined on the basis of a component analysis of the detected spectrum which may allow for a correction of its multiple scatter parts.

According to another exemplary embodiment of the present invention, the pre-processing unit is further adapted for smoothing the first multiple scatter intensities of neighbouring detecting elements of the plurality of detecting elements. This may be performed by, e.g., averaging or calculation of a median.

Thus, scatter of the estimated multiple scatter intensity may be reduced.

According to another exemplary embodiment of the present invention, the examination apparatus is adapted as one of a computer tomography apparatus, a coherent scatter computer tomography apparatus, or an absorption imaging system.

Furthermore, the examination apparatus may comprise a collimator arranged between the radiation source and the detecting elements, wherein the collimator is adapted for collimating the radiation beam emitted by the radiation source to form a fan-beam.

According to another exemplary embodiment of the present invention, the computer tomography apparatus is adapted with detecting elements forming a single-slice detector array or a multi-slice detector array.

The CT/CSCT apparatus according to the invention may be applied as a baggage inspection apparatus, a medical application apparatus, a material testing apparatus or a material science analysis apparatus. A field of application of the invention may be baggage inspection, since the defined functionality of the invention allows a secure and reliable analysis of the content of a baggage item allowing to detect suspicious content, even allowing to determine the type of a material inside such a baggage item.

Such an apparatus or method in accordance with an exemplary embodiment of the present invention may create a high quality automatic system that may automatically recognize certain types of materials and, if desired, trigger an alarm in the presence of dangerous materials.

The radiation source may be adapted for emitting a polychromatic X-ray beam comprising characteristic peaks.

According to another exemplary embodiment of the present invention, a method of examining an object of interest with an examination apparatus may be provided, the method comprising the steps of determining a first multiple scatter intensity in a vicinity of a characteristic peak of the radiation intensity data and correcting the radiation intensity data on the basis of the first multiple scatter intensity.

It is believed that this may allow for an improved correction of intensities measured in an energy-resolved diffraction method without any assumptions on the geometry of the object of interest.

According to another exemplary embodiment of the present invention, an image processing device for examining an object of interest with an examination apparatus may be provided, the image processing device comprising a memory for storing radiation intensity data and a pre-processing unit adapted for carrying out the above-mentioned method steps.

According to another exemplary embodiment of the present invention, a computer-readable medium may be provided, in which a computer program of examining an object of interest with an examination apparatus is stored which, when being executed by a processor, is adapted to carry out the above-mentioned method steps.

The present invention also relates to a program element of examining an object of interest, which, when being executed by a processor, is adapted to carry out the above-mentioned method steps. The program element may be stored on the computer-readable medium and may be loaded into working memories of a data processor. The data processor may thus be equipped to carry out exemplary embodiments of the methods of the present invention. The computer program may be written in any suitable programming language, such as, for example, C++ and may be stored on a CD-ROM. Also, the computer program may be available from a network, such as the WorldWideWeb, from which it may be downloaded into image processing units or processors, or any suitable computers.

It may be seen as the gist of an exemplary embodiment of the present invention, that a change of the spectrum due to multiple scattering is utilized for estimating the fraction of multiple scattered intensity. The characteristic lines of the anode material in the primary spectrum are strongly reduced in intensity in the energy spectrum of multiple scattered radiation due to the energy shift accompanying the Compton scatter process of x-ray photons. This may enable a component analysis of the detected spectrum which may allow for a correction for its multiple scatter part.

These and other aspects of the present invention will become apparent from and elucidated with reference to the embodiments described hereinafter.

Exemplary embodiments of the present invention will be described in the following, with reference to the following drawings.

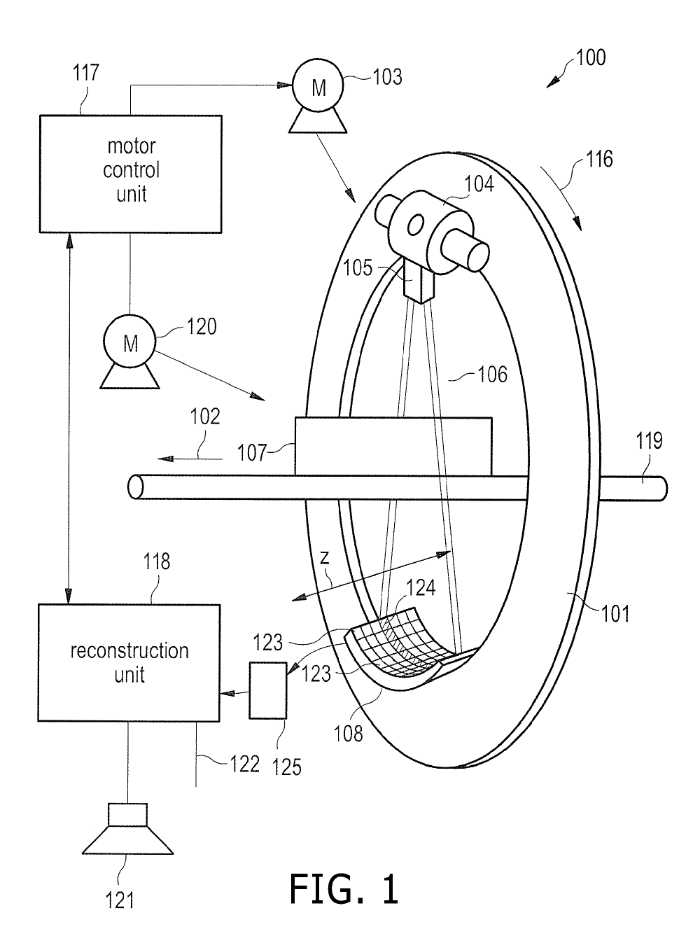

FIG. 1 shows a simplified schematic representation of a CSCT scanner system according to an exemplary embodiment of the present invention.

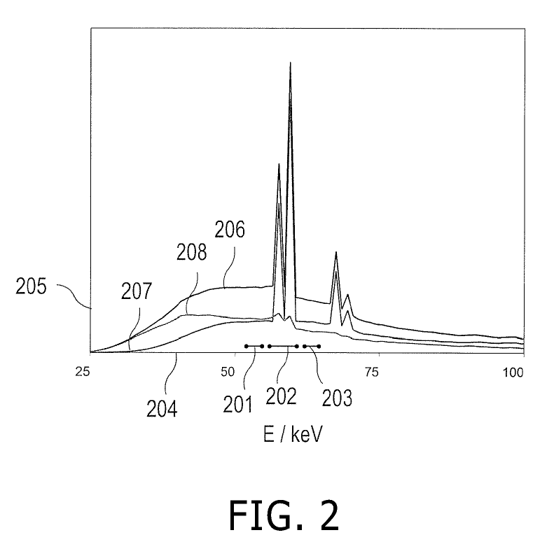

FIG. 2 shows a schematic representation of a decomposition of a Monte-Carlo simulated spectrum of scattered radiation.

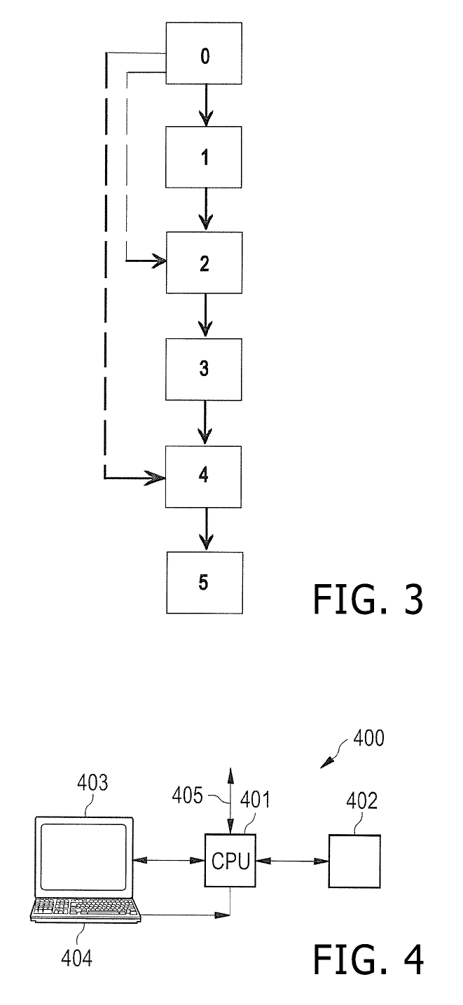

FIG. 3 shows a flow-chart of an exemplary embodiment of a method according to the present invention.

FIG. 4 shows an exemplary embodiment of an image processing device according to the present invention, for executing an exemplary embodiment of a method in accordance with the present invention.

The illustration in the drawings is schematically. In different drawings, similar or identical elements may be provided with the same reference numerals.

FIG. 1 shows an exemplary embodiment of a CSCT scanner system according to an exemplary embodiment of the present invention. With reference to this exemplary embodiment, the present invention will be described for the application in the field of baggage inspection. However, it should be noted that the present invention is not limited to this application, but may also be applied in the field of medical imaging, or other industrial applications, such as material testing.

The computer tomography apparatus 100 depicted in FIG. 1 is a fan-beam CSCT scanner. However, the invention may also be carried out a with a cone-beam geometry. The CSCT scanner depicted in FIG. 1 comprises a gantry 101, which is rotatable around a rotational axis 102. The gantry 101 is driven by means of a motor 103. Reference numeral 104 designates a source of radiation such as an X-ray source, which, according to an aspect of the present invention, emits a polychromatic radiation.

Reference numeral 105 designates an aperture system which forms the radiation beam emitted from the radiation source to a fan-shaped radiation beam 106. The fan-beam 106 is directed such that it penetrates an object of interest 107 arranged in the centre of the gantry 101, i.e. in an examination region of the CSCT scanner, and impinges onto the detector 108. As may be taken from FIG. 1, the detector 108 is arranged on the gantry 101 opposite to the source of radiation 104, such that the length of the detector 108 is covered by the fan-beam 106. The detector 108, which is depicted in FIG. 1, comprises a plurality of detector elements 124 each capable of detecting, X-rays or individual photons which have penetrated the object of interest 107. A second detector next to the fan plane, the scatter detector, measures the radiation scattered out of the fan beam. It also consist of a plurality of detector elements 123 each capable of detecting in an energy resolving manner X-ray or individual photons which have penetrated and scattered inside the object of interest 107. However, the first and the second detector may be arranged as a single detector 108, comprising a middle row of detecting elements 124 for detecting transmitted radiation and multiple rows of detecting elements 123 for detecting scatter radiation.

During a scan of the object of interest 107, the source of radiation 104, the aperture system 105 and the detector 108 are rotated along the gantry 101 in the direction indicated by arrow 116. For rotation of the gantry 101 with the source of radiation 104, the aperture system 105 and the detector 108, the motor 103 is connected to a motor control unit 117, which is connected to a calculation or determination unit 118.

In FIG. 1, the object of interest 107 may be an item of baggage which is disposed on a conveyor belt 119. The conveyor belt 119 may be stopped during the scans to thereby measure single slices. Instead of providing a conveyor belt 119, for example, in medical applications where the object of interest 107 is a patient, a movable table may be used. However, it should be noted that in all of the described cases it may also be possible to perform a circular scan, where there is no displacement in a direction parallel to the rotational axis 102, but only the rotation of the gantry 101 around the rotational axis 102. Furthermore, other scan paths may be performed such as the saddle trajectory by moving the table periodically back and forth at twice the frequency of the source-detector arrangement.

The detector 108 may be connected to the calculation unit 118. The calculation unit 118 may receive the detection result, i.e. the read-outs from the detector elements 123 of the detector 108 and may determine a scanning result on the basis of the read-outs. Furthermore, the calculation unit 118 communicates with the motor control unit 117 in order to coordinate the movement of the gantry 101 with motors 103 and 120 with the conveyor belt 119.

The calculation unit 118, which may comprise the pre-processing unit, may be adapted for constructing an image from read-outs of the detector 108 by determining a first multiple scatter intensity in a vicinity of a characteristic peak of the radiation intensity data, and correcting the radiation intensity data on the basis of the first multiple scatter intensity, according to an exemplary embodiment of the present invention. A reconstructed image generated by the reconstruction unit 118 may be output to a display (not shown in FIG. 1) via an interface 122. However, multiple scatter correction may also be possible in a separate pre-processing unit 125, which may be arranged on the gantry.

The calculation unit 118 may be realized by a data processor to process read-outs from the detector elements 123 of the detector 108.

Furthermore, as may be taken from FIG. 1, the reconstruction unit 118 may be connected to a loudspeaker 121, for example, to automatically output an alarm in case of the detection of suspicious material in the item of baggage 107, or may be connected to a switch triggering a mechanical separation of the item of baggage from other items of baggage.

The coherent scatter computer tomography apparatus 100 for examination of the object of interest 107 includes the detector 108 comprising detecting elements 123 and 124, wherein the plurality of detecting elements 123 is arranged in a matrix-like manner, each being adapted to detect X-rays in an energy-resolved manner. The detecting elements 124 are arranged along a central line and adapted to detect transmitted radiation. Furthermore, the computer tomography apparatus 100 comprises the determination unit or reconstruction unit 118 adapted for reconstructing an image of the object of interest 107.

The computer tomography apparatus 100 comprises the X-ray source 104 adapted to emit X-rays to the object of interest 107. The collimator 105 provided between the electromagnetic radiation source 104 and the detecting elements 124 are adapted to collimate an electromagnetic radiation beam emitted from the electromagnetic radiation source 104. Furthermore, a collimator made of a plurality of lamellae perpendicular to the fan plane and focussed onto the x-ray focal spot may be provided (not depicted in FIG. 1), arranged before the detector 108.

In X-ray imaging, the attenuation of primary rays in the object of interest is measured. In this context both single scattered and multiple scattered radiation is unwanted. In some techniques, anti-scatter grids placed in front of the detector can be used to greatly reduce the amount of scattered radiation reaching the detector. In other techniques, anti-scatter grids are inapplicable. Then, depending on object thickness and size of the illuminated area, the intensity of scatter radiation may exceed the transmitted primary intensity considerably. This is for example described in “Scattered radiation in diagnostic radiology”, H. Chan and K. Doi, Med. Phys. 12(2), 152-165 (1985).

The method according to an exemplary embodiment of the present invention may also be used for absorption imaging to correct for the multiple scattered part, if the requirements are met.

The method according to an aspect of the present invention may allow for a correction of X-ray intensities measured in an energy-resolved diffraction method, for example CSCT, for multiple scatter radiation without any assumptions on the geometry of the object of interest. There may be several approaches to correct the signal measured in absorption imaging from scattered (single and multiple scattered) radiation. However, the method of the present invention provides for a quantification or correction for multiple scatter only.

The method according to an aspect of the present invention may be applied to other X-ray imaging techniques, such as absorption imaging, if they need the requirements which are described below. However, in absorption imaging multiple scattered radiation as well as single scattered radiation is unwanted. Therefore, the method according to the present invention may correct for at least a part of the unwanted signal.

FIG. 3 shows a flow-chart of an exemplary embodiment of a method according to the present invention, which is described in the following with respect to an X-ray imaging set up. The method starts with step 0 in which an acquisition of a primary spectrum and a determination of a multiple scatter spectrum on the basis of the primary spectrum is performed. This is a calibration step which has to be executed only once prior to real measurements. Then, in step 1, a measurement of the X-ray intensities with the detector unit is performed. Then, in step 2, for a plurality of the detector elements or even for each detector element a determination of the multiple scatter intensity near the energy E_{char} of the characteristic line of the anode material is determined by evaluating the measured intensity at and in the vicinity of E_{char}. Here, the primary spectrum and the multiple scatter spectrum derived from the primary spectrum (in step 0) are used for calibration.

Furthermore, in step 3, an averaging of the multiple scatter intensity distribution of neighbouring detector elements may be performed in order to reduce scatter of this signal. However, it should be noted that step 3 is not necessary for carrying out a method according to the present invention.

Then, in step 4, an estimation of the multiple scatter intensities of the full measured energy range from the values at E_{char} and from transmitted intensities is performed for each detector pixel. Again, the multiple scatter spectrum derived in step 0 is used for calibration.

After that, in step 5, a correction of the measured intensities by the estimated multiple scatter intensities is performed, thereby obtaining almost pure single scatter intensities.

These steps will be described in more detail below.

According to an aspect of the present invention, these complex calculations may be performed in real time by a device which may be located near the detector or the measurement data are stored first and the multiple scatter correction may be performed in a computing device after the measurement.

The correction for multiply scattered radiation is based on the fact, that the majority of multiple scattered photons has undergone several Compton scatter processes with a considerable and statistically distributed energy loss. If a conventional X-ray source with a Tungsten anode (or any anode material with high atomic number) is used, the primary spectrum may consist of the continuous bremsstrahlung and the characteristic lines of the anode material. Due to energy shift accompanying the Compton process, multiply scattered photons form a spectrum with considerably lower relative intensities at the characteristic energies. This is depicted in FIG. 2.

FIG. 2 shows a schematic representation of a decomposition of a Monte-Carlo simulated spectrum of scattered radiation.

The horizontal axis 204 shows the energy of the scattered radiation in units keV. The vertical axis 205 shows the corresponding scatter intensities in arbitrary units.

Curve 206 shows the total scatter intensity, curve 207 shows the single scatter intensity and curve 208 shows the multiple scatter intensity.

A material with a very smooth scatter function has been implemented in the simulation. So, no scatter peaks occur and the scatter spectrum resembles the primary spectrum.

As stated above, due to energy shift accompanying the Compton process, multiply scattered photons form a spectrum with considerably lower relative intensities at the characteristic energies (as depicted in FIG. 1). In contrast, transmitted and, because of small scatter angles, also single scattered radiation undergoes no considerable energy shift and therefore the relative intensity of the characteristic lines is equal to the value in the primary spectrum (see curve 207 of FIG. 1). This fact is used to determine the amount of multiple scattered radiation.

The proposed method may also work with x-ray spectra consisting of a continuous spectrum and sharp peaks, like spectra from electron impact sources.

To be able to apply the correction, the x-radiation to be corrected may have to be measured with a high spectral resolution.

The penetrated thickness of the object may have to be estimated. One way to do so is to measure the attenuation of primary radiation in addition to the scatter intensities.

Calibrations (Step 0 in FIG. 3)

The technique makes use of the characteristic lines in the primary spectrum emerging from the x-ray source. For the measurement of its intensity three regions of interest in the energy spectrum have to be defined: two energy intervals away from the characteristic line—one below and one above the characteristic energies of the anode material—and the third energy interval including characteristic energies. This is depicted in FIG. 2.

The intensity of the characteristic line relative to the bremsspectrum in the primary spectrum has to be evaluated according to

$${u}_{prim} = \frac{{I}_{prim} ( {E}_{char} )}{{I}_{prim} ( {E}_{char} \pm Δ E )} .$$

Here Iₚᵣᵢₛₘ(E_{char}) is the mean intensity of the primary spectrum in the energy interval containing the characteristic energy 202 and Iₚᵣᵢₛₘ(E_{char}±ΔE) is the mean intensity in the other two energy intervals 201 and 203 in FIG. 2.

To be able to recalculate the spectral distribution of multiple scattered radiation, its spectrum I_{MS,std} has to be measured or simulated with a standard object.

A value analogous to uₚᵣᵢₘ can be found for this spectrum of multiple scattered radiation:

$${u}_{MS} = \frac{{I}_{MS} ( {E}_{char} )}{{I}_{MS} ( {E}_{char} \pm Δ E )}$$

Due to the stated effects this value is nearly 1.

These measurements and calculations have to be done only once while the following steps have to be executed during or after the data acquisition.

Calculations of I_{MS}-intensities at characteristic energy (Step 2 in FIG. 3)

From measured data the relative intensity of characteristic lines have to be calculated of each energy resolving pixel i of the detector:

$${u}_{meas}^{i} = \frac{{I}_{meas}^{i} ( {E}_{char} )}{{I}_{meas}^{i} ( {E}_{char} \pm Δ E )}$$

Presuming, that intensity variations due to scatter peaks have no considerable influence on uₘₑₐₛⁱ, the multiple scatter intensity in the energy interval containing the characteristic energy can be calculated from these values using uₚᵣᵢₘ (from the primary spectrum) and u_{MS} (from the reference multiple scatter spectrum):

$${I}_{MS}^{i} ( {E}_{char} ) = \frac{{u}_{MS}}{{u}_{meas}^{i}} \frac{{u}_{prim} - {u}_{meas}^{i}}{{u}_{prim} - {u}_{MS}} {I}_{meas}^{i} ( {E}_{char} )$$

Smoothing and Interpolation (Step 3)

Probably in some detector pixels i scatter peaks interfere with the calculation of the multiple scatter intensity. Therefore, and since the intensity distribution of multiple scattered radiation is rather smooth, it is advantageous to smooth the multiple scatter estimates from the former equation for neighboring pixels, e.g., by calculating median values. The result is a smoothed estimate I_{MS,smooth}ⁱ(E_{char}) for the multiple scatter intensity at the characteristic energies for all detector pixels.

Extrapolation to Full Energy Spectrum (Step 4)

If the full energy-resolved output of the detector is to be corrected for its multiple scatter part, the spectral intensity of the multiple scatter has to be calculated for the full energy range. To do so, the energy spectrum of multiple scattered radiation measured in advance is adapted to the attenuation measured with each detector pixel:

The thickness of the transmitted material has to be calculated from the of ratio of transmitted to primary radiation:

$$d = \frac{- 1}{{μ}_{abs}} ln ( \frac{{I}_{primary}}{{I}_{0}} )$$

Here, μ_{abs} is the attenuation coefficient of the mean energy of the primary spectrum.

Now the whole multiple scatter spectrum can be estimated:

$${I}_{MS}^{i} ( E ) = \frac{{I}_{MS , smooth}^{i} ( {E}_{char} )}{{I}_{MS , std} ( {E}_{char} , {d}_{0} ) exp ( - {μ}_{MS} ( {E}_{char} ) ( d - {d}_{0} ) )} \cdot {I}_{MS , std} ( E , {d}_{0} ) \cdot exp ( - {μ}_{MS} ( E ) ( d - {d}_{0} ) )$$

Here d₀ is the material thickness of the phantom that was used in the measurement or simulation of the standard multiple scatter spectrum I_{MS,std}. And μ_{MS}(E) describes the energy dependence of attenuation of multiple scattered radiation, which can be described by

$${μ}_{MS} ( E ) = ( C - 0.0004 \frac{E}{keV} ) \frac{1}{cm} .$$

Correction of the Measured Spectra (Step 5)

To correct the measured intensities for multiple scatter radiation, the calculated multiple scatter intensities have to be subtracted:

I_{corrected}ⁱ(E)=Iₘₑₐₛⁱ(E)−I_{MS}ⁱ(E)

The invention may applied in all x-ray scattering and imaging techniques, which fulfil the demands on the x-ray spectrum used and on the detector.

FIG. 5 depicts an exemplary embodiment of an image processing device according to the present invention for executing an exemplary embodiment of the method in accordance with the present invention. The image processing device 400 depicted in FIG. 5 comprises a central processing unit (CPU) or image processor 401 connected to a memory 402 for storing an image depicting an object of interest, such as a patient or an item of baggage. The data processor 401 may be connected to a plurality of input/output network for diagnosis devices, such as a CSCT device. The data processor 401 may furthermore be connected to a display device 403, for example, a computer monitor, for displaying information or an image computed or adapted in the data processor 401. An operator or user may interact with the data processor 401 via a keyboard 404 and/or other output devices, which are not depicted in FIG. 5. Furthermore, via the bus system 405, it may also be possible to connect the image processing and control processor 401 to, for example, a motion monitor, which monitors a motion of the object of interest. In case, for example, a lung of a patient is imaged, the motion sensor may be an exhalation sensor. In case the heart is imaged, the motion sensor may be an electrocardiogram.

In any X-ray diffraction method only the single scattered photons carry valuable information whereas multiple scattered radiation degrades data quality. According to an aspect of the present invention, image quality may be improved by quantification of the multiple scattered intensity and a subtraction strategy. This may be provided by using an electron impact X-ray source and an energy-resolving detector, because spectral information in the vicinity of the characteristic peaks is evaluated to quantify the amount of multiple scattered radiation. Although the use of coherent scatter computer tomography has been described in detail, the method is also applicable to other X-ray techniques. Multiple scatter radiation may lead to artefacts in reconstructed CSCT data. Its correction may avoid these artefacts and therefore may lead to a considerable improvement of the measured output.

Exemplary embodiments of the invention may be sold as a software option to CT scanner console or as a separate pre-processing unit adapted for pre-processing (and thus correcting) the detected signals before reconstruction by a reconstruction unit.

It should be noted that the term “comprising” does not exclude other elements or steps and the “a” or “an” does not exclude a plurality and that a single processor or system may fulfill the functions of several means or units recited in the claims. Also elements described in association with different embodiments may be combined.

It should also be noted, that any reference signs in the claims shall not be construed as limiting the scope of claims.

1. An examination apparatus (100) for examination of an object of interest (107), the examination apparatus (100) comprising:
a radiation source (104) adapted for emitting polychromatic electromagnetic radiation comprising at least one characteristic peak;
a detector unit having at least one detecting element (123) for acquisition of radiation intensity data in an energy resolving manner;
a pre-processing unit (125), the pre-processing unit (125) being adapted for:
determining a first multiple scatter intensity in an energy interval in the vicinity of an energy interval contain the characteristic peak of the radiation intensity data;
correcting the radiation intensity data on the basis of the first multiple scatter intensity.

2. The examination apparatus (100) of claim 1,
wherein the detector unit comprises a first detecting element (123) and a second detecting element (123); and
wherein the determination is performed for the first detecting element (123) and the second detecting element (123).

3. The examination apparatus (100) of claim 1,
wherein the detector unit further comprises third detecting elements (124) for acquisition of transmitted intensity data;
wherein the correction is performed on the basis of an estimation of a second multiple scatter intensity of a full measured energy range for each one of the first detecting elements and the second detecting elements; and
wherein the estimation is performed on the basis of the first multiple scatter intensity and the transmitted intensity data.

4. The examination apparatus (100) of claim 2,
wherein the pre-processing unit (125) is further adapted for smoothing the first multiple scatter intensities of neighbouring detecting elements of the plurality of detecting elements (123).

5. The examination apparatus (100) of claim 1,
wherein the examination apparatus (100) is adapted as one of a computer tomography apparatus, a coherent scatter computer tomography apparatus, an absorption imaging system, or an x-ray scattering examination apparatus.

6. The examination apparatus (100) of claim 1, further comprising:
a collimator (105) arranged between the radiation source (104) and the detecting elements (123);
wherein the collimator (105) is adapted for collimating the electromagnetic radiation beam emitted by the radiation source (104) to form one of a fan-beam or a cone-beam.

7. The examination apparatus (100) of claim 1, wherein the detecting elements (123, 124) form a single-slice detector array.

8. The examination apparatus (100) of claim 1, wherein the detecting elements (123, 124) form a multi-slice detector array (108).

9. The examination apparatus (100) of claim 1, configured as one of the group consisting of a baggage inspection apparatus, a medical application apparatus, a material testing apparatus and a material science analysis apparatus.

10. The examination apparatus (100) of claim 1, wherein the radiation source (104) is adapted for emitting a polychromatic x-ray beam.

11. A method of examining an object of interest (107) with an examination apparatus (100), the examination apparatus (100) comprising a radiation source (104) adapted for emitting polychromatic electromagnetic radiation comprising at least one characteristic peak, a detector unit having at least one detecting element (123) for acquisition of radiation intensity data in an energy resolving manner, and a pre-processing unit (125), the method comprising the steps of:
determining a first multiple scatter intensity in an energy interval in the vicinity of an energy interval containing the characteristic peak of the radiation intensity data; and
correcting the radiation intensity data on the basis of the first multiple scatter intensity.

12. An image processing device for examining an object of interest (107) with an examination apparatus, the image processing device comprising:
a memory for storing energy resolved radiation intensity data;
a pre-processing unit (125), being adapted for:
determining a first multiple scatter intensity in an energy interval in the vicinity of an energy interval containing the characteristic peak of radiation intensity data; and
correcting the radiation intensity data on the basis of the first multiple scatter intensity.

13. A computer-readable medium (402), in which a computer program of examining an object of interest (107) with an examination apparatus (100) is stored which, when being executed by a processor (401), is adapted to carry out the steps of:
determining a first multiple scatter intensity in an energy interval in the vicinity of an energy interval containing the characteristic peak of radiation intensity data; and
correcting the radiation intensity data on the basis of the first multiple scatter intensity.

14. A program element of examining an object of interest (107), which, when being executed by a processor (401), is adapted to carry out the steps of:
determining a first multiple scatter intensity in an energy interval in the vicinity of an energy interval containing the characteristic peak of energy resolved radiation intensity data; and
correcting the radiation intensity data on the basis of the first multiple scatter intensity.
